(19) United States
(12) Patent Application Publication
(10) Pub. No.: US 2013/0060203 A1
(43) Pub. Date: Mar. 7, 2013

(54) SYRINGE WITH YIELDING ELEMENT

(71) Applicant: Jan Svensson, Huskvarna (SE)

(72) Inventor: Jan Svensson, Huskvarna (SE)

(21) Appl. No.: 13/696,467

(22) PCT Filed: May 2, 2011
(86) PCT No.: PCT/SE11/00076

§ 371 Date: Nov. 6, 2012

(30) Foreign Application Priority Data
May 6, 2010 (SE) 1000466-1

(51) Int. Cl. A61M 5/315

(52) U.S. Cl. 604/222; 604/218

(57) ABSTRACT

A syringe includes a cylindrical container in which is arranged at least a first piston which delimits a storage volume. The syringe is provided with at least one yielding element having a first side that delimits the storage volume in a liquid tight fashion, and having a second side that is in communication with the surroundings of the syringe. This renders the syringe liquid tight even if the pressure of the substance in the storage volume increases. This may be used when sterilizing with heat or when freeze drying substance in the syringe. The syringe can include a first piston with a first piston rod and a second piston with a second piston rod. The two pistons then define two storage volumes which may be used for storage of freeze dried substance and a solvent for the freeze dried substance, respectively. The different substances are unequally affected by heat sterilization, which may give rise to a pressure difference between the storage volumes, but the yielding element compensates for this such that no leaks between them occur.

### BACKGROUND AND SUMMARY

The present invention relates to a syringe with a yielding element.

In particular, it relates to such a syringe with a yielding element where the syringe is a multi chamber syringe.

Multi chamber syringes are known through for example WO2008150208 which discloses a syringe with several pistons that delimit several storage volumes in the syringe. The syringe may be used for storing several substances separate from each other, which are activated Only once they are mixed, and where the substances last longer separately that when mixed. A typical application is storage of a freeze dried substance in a first storage volume and a solvent, for example a saline water solution, in a second storage volume.

As long as the substances are kept apart, freeze dried substances last a very long time and in the design a large effort has been put into keeping the substances apart up until the mixing instance. When the contents are to be used the substances must be mixed, and when pressure exceeding a set limit is manually applied to the multi chamber syringe, substance flows from one to the other storage volume such that the substances may mix. The mix must then be injected within a limited time span in order to remain active.

Several different processes in the filling, process do however achieve a pressure difference between the storage volumes, which inadvertently may cause leakage between the storage volumes. Two typical such processes in the filling process are heat sterilization and freeze drying. During heat sterilization vapour pressure of the liquids in the storage volumes increases and if two consecutive storage volumes contain and do not contain liquid, respective, a pressure difference between the storage volumes may occur. By freeze drying vacuum is applied, which may cause a corresponding pressure difference and in addition water expands when freezing which may give rise to a corresponding effect and cause leakage.

It is desirable to provide a syringe with a yielding element which minimizes the risk of leakage when heat sterilizing or when freeze drying.

The invention, according to an aspect thereof, relates to a syringe comprising a cylindrical container 1 in which is arranged at least a first piston 5, 5a-b which delimits at least one storage volume. The syringe is provided with at least one yielding element 16a-b having a first side that delimits the storage volume in a liquid tight fashion, and having a second side that is in communication with the surroundings of the syringe. This renders the syringe liquid tight even if the pressure of the substance in the storage volume increases. This may advantageously be used when sterilizing with heat or when freeze drying substance in the syringe. In one embodiment the yielding element 16a-b is constituted by a bellows.

The invention, according to an aspect thereof, thus relates to a closed, sterile injection syringe with at least two separate chambers containing substances intended to be mixed before injection. The liquid that has be enclosed in a closed, separate chamber must be allowed to expand during heat sterilization and/or a freeze drying process without the enclosed liquid or the interior of the chamber being contaminated or the syringe blown apart by the increased interior pressure during expansion. This is advantageously rendered possible by the syringe according to an aspect of the invention.

In a particularly advantageous embodiment the syringe comprises at least a first 5a piston with a first piston rod 12 and a second piston 5b with a second piston rod 13, where the first piston rod 12 and the second piston rod 13 may be locked together with a locking element 15. The two pistons then define two storage volumes which advantageously may be used for storage of freeze dried substance and a solvent for the freeze dried substance, respectively. Different substances are very differently affected by heat sterilization, which may give rise to a pressure difference between the storage volumes, but the yielding element compensates for this such that no leaks between them occur.

In yet another particularly advantageous embodiment the second piston 5b is provided with a sealing element 7b which in an unloaded state is sealing, while when loaded allows fluids to pass in a first direction, but when loaded disallows fluids to pass in as second direction. This allows for later mixing of the substances, but keeps them separated during storage, sterilization and freeze drying.

During heat sterilization the vapor pressure increases in the upper storage volume such that vapor or liquid may be forced past the sealing ring, which gives a reduction of the intended volume of liquid and may give rise to an improper mixing ratio. This is prevented by the yielding element.

### BRIEF DESCRIPTION OF THE DRAWINGS

FIG. 1 shows a longitudinal cross section of a prior art two Chamber syringe

FIG. 2 shows a cross section orthogonal to the longitudinal axis of a prior art two chamber syringe

FIG. 3 shows a fully assembled and filled syringe according to an aspect of the invention

FIG. 4 shows a cross section through a syringe according to an aspect of the invention in a first step of the filling process

FIG. 5 shows the upper end of the syringe with a locking device

FIG. 6 shows an expandable plug for use with the syringe according to an aspect of the invention

FIG. 7 shows a mouth piece for use with the syringe according to an aspect of the invention

FIG. 8 shows a cross section through a syringe according to the an aspect of invention in a second step of the filling process

FIG. 9 shows a cross section through a syringe according to an aspect of the invention in a third step of the filling process

FIG. 10 shows the mouth of the syringe in greater detail

FIG. 11 shows the upper piston of the syringe with bellows in greater detail

FIG. 12 shows the syringe during mixing of the substances in the chambers

FIG. 13 shows the lower piston of the syringe in greater detail

FIG. 14 shows the upper end of the syringe with a locking device

FIG. 15 shows the syringe when the substances in the chambers have been mixed.

FIG. 16 shows the upper piston of the syringe with bellows in greater detail

FIG. 17 shows the syringe during dispensing of mixed substance

FIG. 18 shows pan of the upper piston of the syringe with bellows in greater detail

FIG. 19 shows the upper piston and the outer piston rod separately

FIG. 20 shows the grip 14 for the inner piston rod separately

FIG. 21 shows a locking element 15 for the inner piston rod separately

### DETAILED DESCRIPTION

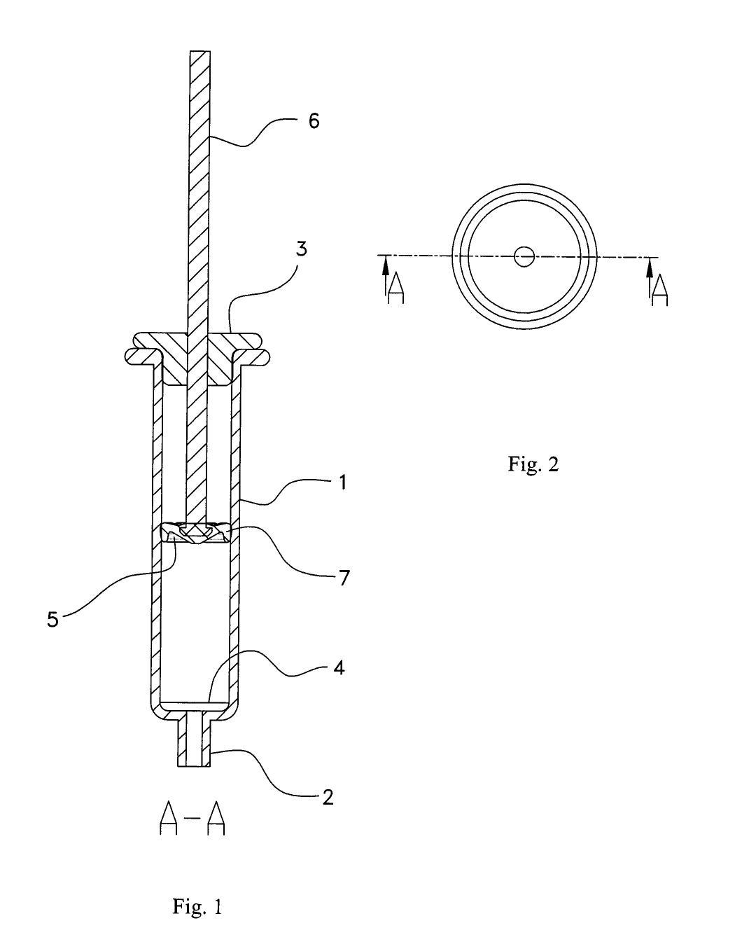

FIG. 1 shows a longitudinal cross section of a prior art two chamber syringe. The two chamber syringe comprises an essentially cylindrical container 1 with a spout 2 at the lower end and an upper seal 3 at the upper end. At the spout sits a lower seal 4 and in the volume between the upper 3 and lower 4 seals is a storage volume for substances for medical use.

The storage volume is separated by a piston 5 into an upper storage volume and a lower storage volume. The piston is surrounded along its periphery by a sealing ring that normally holds the substances in the two storage volumes separated. The sealing ring is embodied to let liquid pass in one direction at a relatively low pressure difference, but block liquid flow in the other direction even at a high pressure difference, as disclosed by for example SE0900798-0.

A user may apply force to the piston with a piston rod 6 which extends through a sealed opening in the upper seal 3. By pulling the piston upwards the substance in the upper storage volume is forced past the sealing ring and is mixed with the substance in the lower storage volume. Thereafter, the user may push the rod downwards and dispense the mixed substances through the spout.

In a typical application, substance is freeze dried in the lower storage volume and a saline water solution is stored in the upper storage volume. As long as the used does not apply force to the piston rod, the two substances are kept apart and the dry, freeze dried substance then lasts for a long time. During freeze drying the water in the upper storage volume may freeze and then expands. During the expansion, ice may force itself past the sealing ring and once molten mixes with the freeze dried substance that then starts to decay. During heat sterilization the vapor pressure instead increases in the upper storage volume such that vapor or liquid may be forced past the sealing ring.

FIG. 2 shows a cross section orthogonal to the longitudinal axis of a prior art two chamber syringe. The figure shows a cross section through the cylindrical container 1 and illustrates an in cross section ring shaped element.

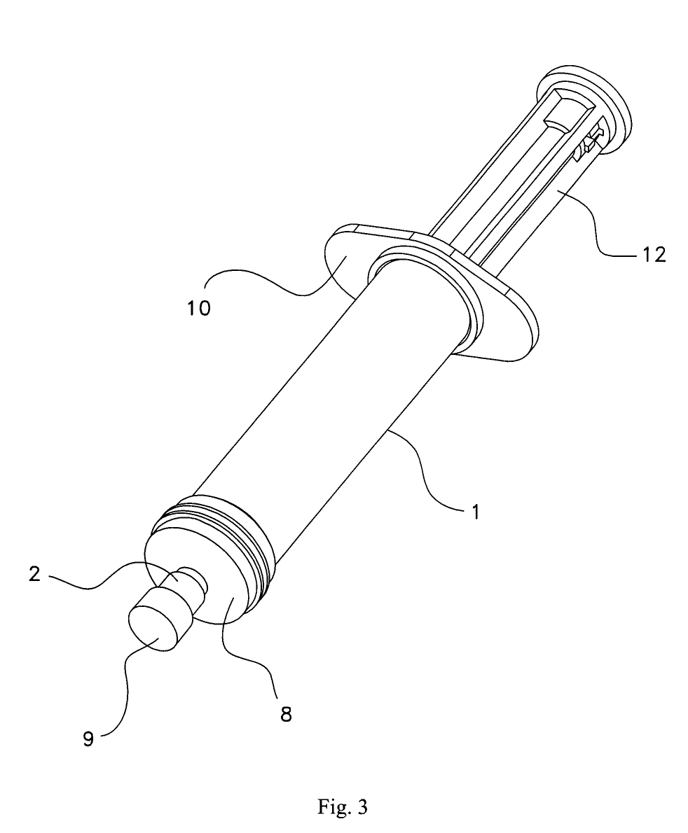

FIG. 3 shows a fully assembled and filled syringe according to the invention. The syringe comprises a cylindrical container 1, typically made from glass or a semi transparent polymer, but the illustration does not show the interior details of the syringe but only its outer surfaces. At the lower end of the container sits a spout 2 on a mouth piece S which is attached to the open lower end of the cylindrical container in a watertight fashion. The spout 2 is provided with a lid 9 such that the lower end is completely sealed after filling.

At the other end of the cylinder a handle 10 sits that the user may use to press against when the piston rod is to be pushed or pulled in either direction. In the interior of the cylinder two piston rods extend, one within the other, of which only the outer piston rod 12 is visible. With the syringe filled and sterilized, typically containing at least a freeze dried substance, the syringe may be stored for a long time without the contents deteriorating. Thereafter the substances may be mixed and then dispensed in the fashion described in conjunction with FIGS. 12-18. During dispensing for injection the syringe may be provided with an injection needle or attached to a tube of intravenous supply of substance o the circulatory system.

FIG. 4 shows a cross section through a syringe according to the invention in a first step of the filling process. The cylindrical container 1 is in this step open at its lower end. An upper piston 5a and a lower piston 5b are arranged in the cylindrical container. The two pistons delimits an upper and a lower storage volume, where the upper storage volume extends between the pistons, while the lower storage volume extends between the lower piston 5b and the lower end of the cylinder. The upper storage volume is filled with a saline water solution, which is indicated by the dashes in this volume, while the lower storage volume remains empty.

An outer piston rod 12 is attached to the upper piston 5a and an inner piston rod 13, extending inside the outer, is attached to the lower piston. The inner piston rod is slideably but sealingly arranged within the outer, such that the piston rods may slide with respect to each other without substance of air leaking in between the piston rods.

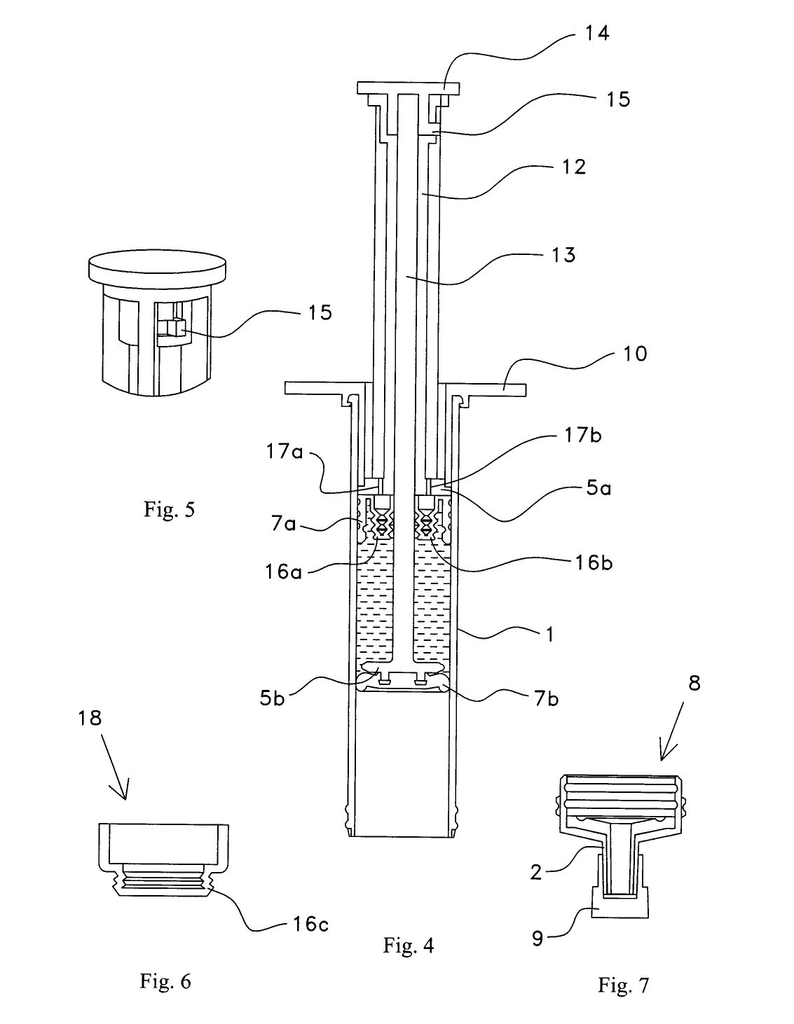

The inner piston rod is at the end that faces away from the lower piston provided with a grip 14 that facilitates pulling or pushing the lower piston. In the illustrated first stage of the tilling process, the piston rods are locked together with a lock 15 that is more clearly illustrated in FIG. 5.

The lower piston is provided with a sealing ring 7b of a kind disclosed in SE0900798-0. The sealing ring extends to the inner surface of the cylindrical container 1 and normally seals against it, but unloaded its outermost part curves downwards. This makes it allow liquid to flow from the upper to the lower storage volumes at a low pressure difference between the two, while a substantially higher pressure is needed to make the liquid flow past the sealing ring from the lower to the upper storage volumes. The sealing ring is designed to prevent, at any normally occurring pressure differences, liquid from flowing from the lower to the upper storage volume, while the opposite is allowed only when a used applies a sufficient force to achieve such a flow.

The upper piston 5a comprises a seal 7a that seals against the inner surface of the cylinder and is not designed to preferentially allow liquid to pass in one direction, but is intended to seal all the time. The upper piston has in its centre an opening through which the inner piston rod extends and the piston seals against the inner piston rod. Two bellows 16a-b are arranged on the upper piston which are sealingly attached to the piston, but may expand or compress. The bellows constitute an element kind that in the claims for simplicity is denoted yielding elements, but the denotation is not intended to be delimiting and refers to expandable and/or compressible elements.

The first 16a and second 16b bellows are hollow and approximately cylindrical with a closed lower end and an open upper end. The approximately cylindrical outer surfaces of the bellows are accordion like with a series of protruding ring shaped elements separated by a series of ring shaped elements of smaller radius. In the figure, the bellows are illustrated in cross section and shows as having zig-zag shaped right and left sides. The accordion like design facilitates expansion and compression of the bellows during varying pressure differences between the insides and outsides of the bellows.

The interior of the bellows is in communication with the exterior of the syringe via conduits 17a-b, that extend to the inner cavity of the bellows via their open upper surfaces. If a pressure difference between the upper storage volume and the surroundings of the syringe occurs, or if the substance in the upper storage volume expands or is compressed, the bellows may expand or compress to compensate for this pressure difference. The bellows are made from a resilient but yielding substance, such as for example rubber which tends to return to its unloaded state. In the figure there is no pressure difference between the upper storage volume and the surroundings of the syringe, so the bellows have adopted the shape they have when unloaded.

FIG. 5 shows the upper end of the syringe with a locking device that hold the piston rods locked together. The lock may be opened such that the piston rods and thereby the pistons may move in relation to each other. When delivered to a customer, the lock is in its locked position and therefore normally prevents the pistons to move with respect to each other, such that the substances in the storage volumes are not inadvertently mixed. The inner piston rod comprises a locking element 15 that extends perpendicularly to the longitudinal extension of the piston rod and out through a groove in the inner piston rod. The groove extends part of the circumference of the outer piston rod and attaches at one of its ends to a groove that extends upwards and is open against the upper surface of the outer piston rod. By turning the inner piston rod around its centre axis, the locking element 15 may be turned between a position in the groove that does not reach the groove that extends upwards, and an opposite position where the locking element 15 is positioned under the groove that extends upwards. The rods are in these positions locked together and moveable in the axial direction with respect to each other, respectively.

FIG. 6 shows an expandable plug for use with the syringe according to the invention when the lower storage volume too is to be heat sterilized. The plug is intended to be attached to the lower opening of the cylindrical container 1 such that it is sealed. The plug does in its lower end comprise a third bellows 16c of essentially the same embodiment as the first 16a and second 16b ones. If the second storage volume is to be heat sterilized, the third bellows 16c may expand and compensate for the pressure increase such that no leakage between the storage volumes occurs.

FIG. 7 shows a mouth piece for use with the syringe. The mouth piece is received on the lower opening of the cylindrical container 1 such that the container is sealed in the same way as by the expandable plug. The mouth piece does at its lower end comprise a spout 2 that is sealed with a lid 9.

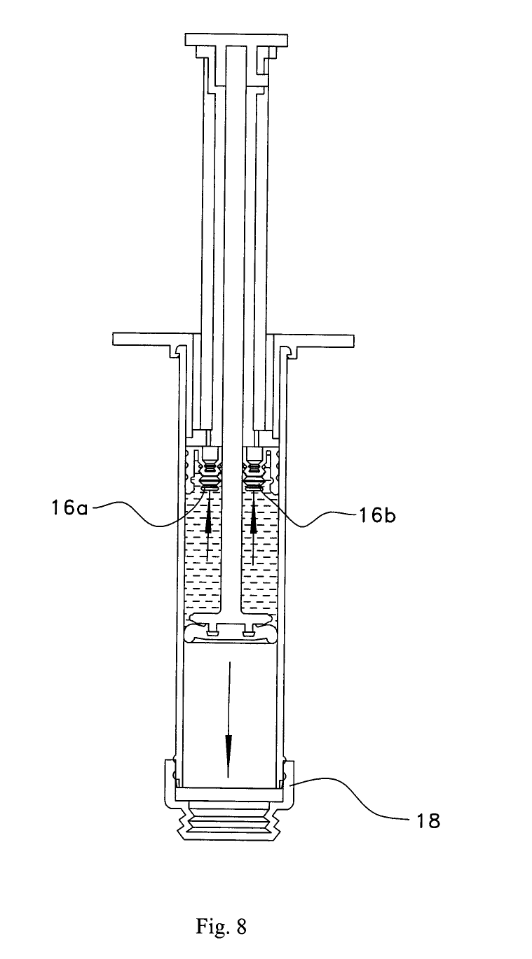

FIG. 8 shows a cross section through a syringe according to the invention in a second step of the filling process. In the second step the syringe is heat sterilized and the contents of both storage volumes tend to expand as a result of the increased pressures. The pressure increases give rise to forces that are illustrated by the arrows in the figures. The pressure in the upper storage volume gives rise to a force acting on the first and second bellows 16a-b and the figure illustrates how these have been compressed to compensate for the pressure increase. The syringe is at its lower end closed by the expandable plug 18 and the third bellows 16c has expanded to compensate for the pressure increase in the lower storage volume.

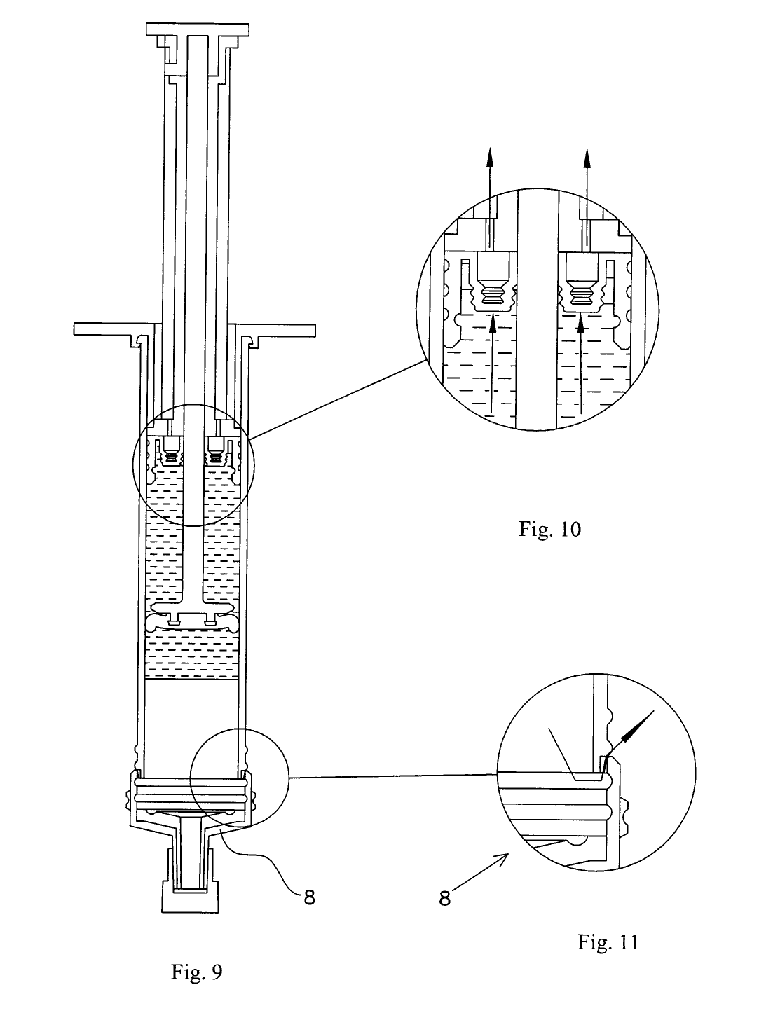

FIG. 9 shows a cross section through a syringe according to the invention in a third step of the filling process, when freeze drying of the substance in the lower storage volume is ongoing. During freeze drying the pressure difference between the storage volumes changes and in addition the water in the upper storage volume may freeze and expand.

FIG. 11 shows the upper piston of the syringe in greater detail with the first and second bellows that in this stage have been compressed to compensate for liquid in the upper storage volume having frozen and expanded. The expansion of the liquid and the compression of the bellows is indicated by the two lower arrows in the figure.

In this stage, the mouth piece 8 instead of the expandable plug 18 is attached to the lower opening of the cylindrical container 1 and FIG. 10 shows the mouth of the syringe in greater detail at this stage. The lower storage volume is here filled with an active substance in solution that is being freeze dried by applying vacuum to the syringe. In order to let vaporized solvent leave the lower storage volume, a aeration conduit is arranged between the mouth piece 8 and the lower opening of the cylindrical container 1 through which vapour may pass, as indicated by the arrow in the figure. After this stage the syringe is filled, sterilized, the dry substance freeze dried and the syringe is ready for storage for later use.

In FIG. 12 a user has initiated use of the syringe and shows the syringe during mixing of the substances in the chambers. The two piston rods have unlocked from each other using the lock 15 and the inner piston rod 13 has been pulled upwards from its position during storage of the syringe. The upper piston is the forced upwards in relation to its position during storage of the syringe and liquid from the upper storage volume is forced past the upper piston and into the lower storage volume as is indicated by the arrows in the figure.

FIG. 13 shows the lower piston of the syringe in greater detail, where the outer portion of the sealing ring, 7b has flexed downwards and towards its centre, such that liquid may pass from above and downwards. In the figure the yet freeze dried substance is denoted as element 19 while the liquid from the upper storage volume is dashed.

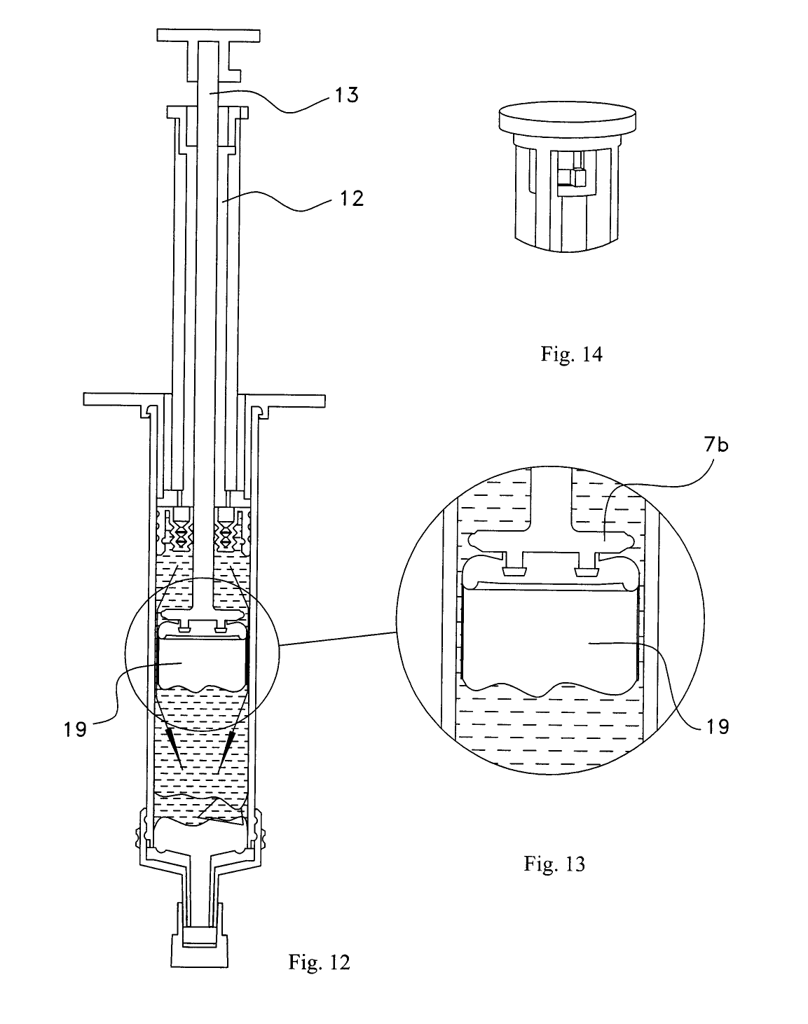

FIG. 14 shows the upper end of the syringe with a locking device, here in an unlocked position such that the two pistons may move with respect to each other.

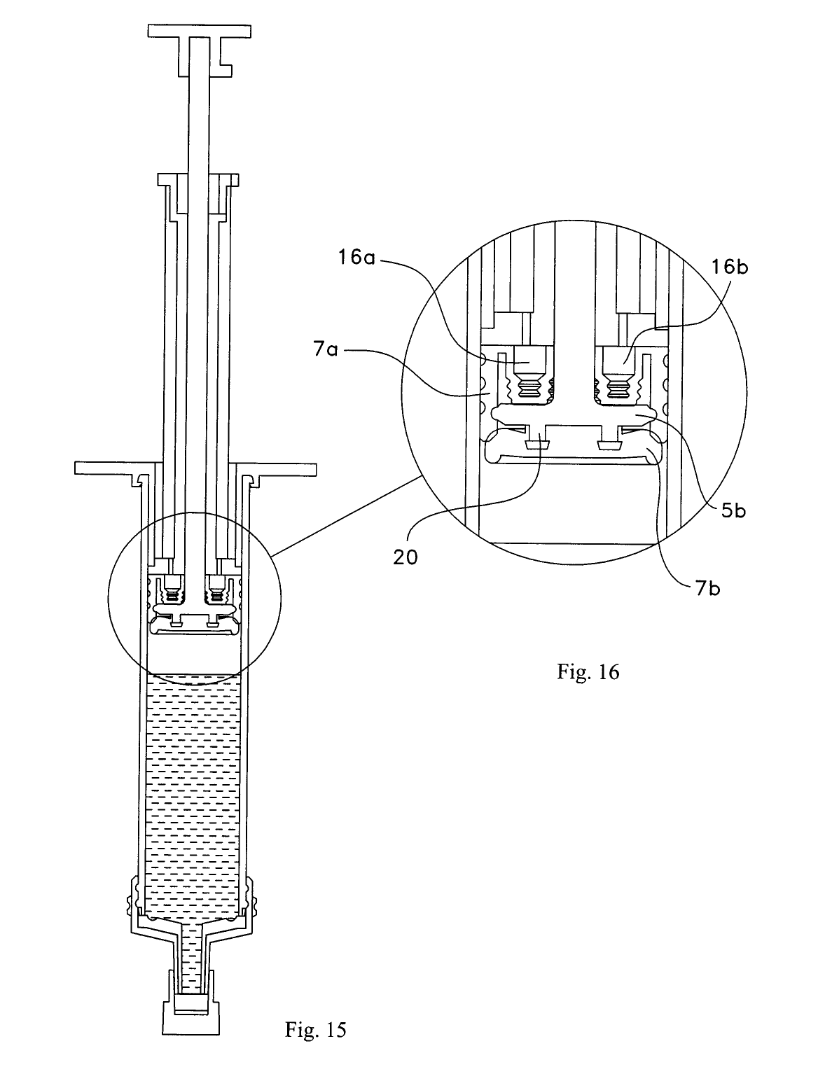

FIG. 15 shows the syringe when the substances in the chambers have been mixed and the lower piston has reached the upper. In this position the pistons are locked together with a piston lock which is more clearly illustrated in FIG. 16. The purpose of the piston lock is to allow the user to then push down the handle 14 of the inner piston rod only, and still jointly push both pistons towards the mouth. In this position the upper surface of the lower piston is immediately adjacent the lower surface of the upper piston.

FIG. 16 shows the upper piston of the syringe in greater detail with the bellows after mixing, when both pistons are locked together. The upper piston 7a comprises a cylindrical opening that is directed downwards and may receive the lower piston. The lower piston 5b comprises a comparatively rigid, cylindrical plate with attachment knobs 20 that are inserted into the sealing ring 7b and holds it attached to the underside of the cylindrical plate. In the figure the cylindrical plate has been receive in the cylindrical opening on the upper piston 7a and the cylindrical plate extends around its periphery into a corresponding receiving groove on the inner surface of the cylindrical opening. Cooperation between the cylindrical plate and the receiving groove is what locks the two pistons together.

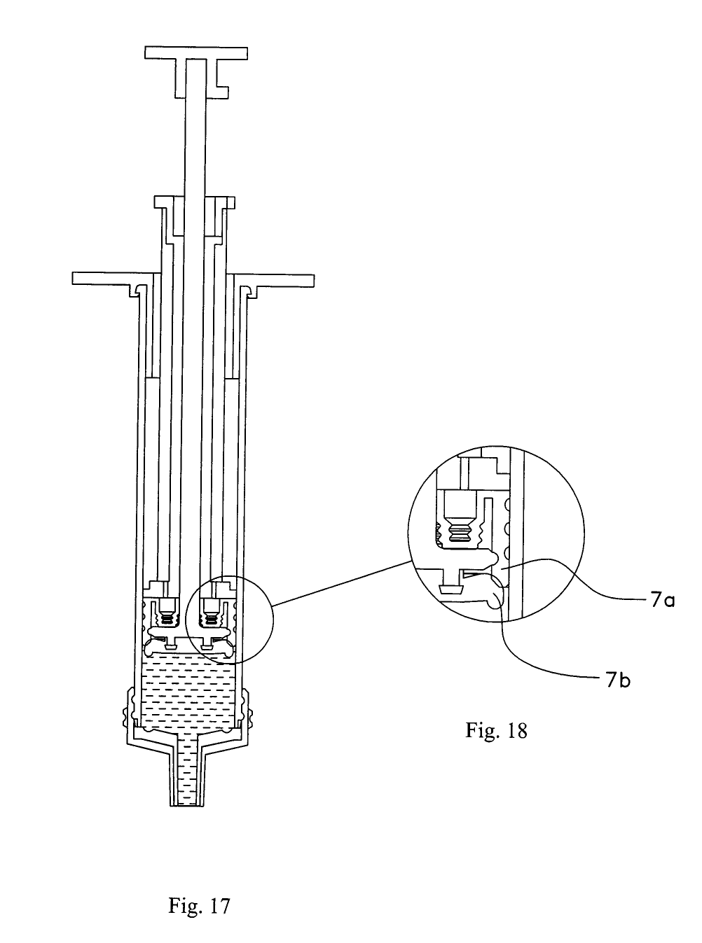

FIG. 17 shows the syringe during dispensing of mixed substance and the piston pair is now being pushed downwards. The sealing ring 7b on the lower piston is designed to resist passage of liquid from below and upwards, which also means that it resists or counteracts movement of the sealing ring in the downward direction it is now moving. This is avoided by a constructional feature illustrated more clearly in FIG. 18.

FIG. 18 shows part of the upper piston of the syringe in greater detail with one of the bellows. When the cylindrical plate has been received into the receiving groove on the cylindrical opening of the upper piston, the outer portion of the sealing ring reaches the lower end of the upper piston. The lower end of the upper piston extends around the inner surface of the cylindrical container 1 and does in this position force the outer portion of the sealing ring to bend somewhat downwards and therefore deviate from the inner surface of the cylindrical container. In this way the sealing ring is no longer aligned against the inner surface of the cylindrical container 1 and the piston pair easily slides downwards for dispensing of mixed substances. In the figure the sealing ring and the lower end of the upper piston are illustrated are being overlapping, but this is only in order to illustrate why the sealing ring bends away.

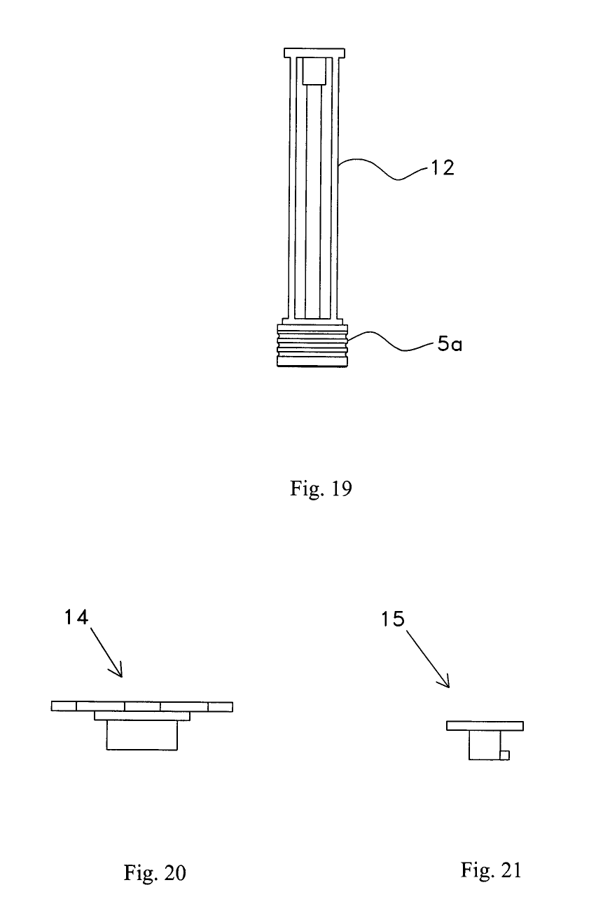

FIG. 19 shows the upper piston and the outer piston rod separately. The figure is not in cross section, so the bellows are not visible. FIG. 20 shows the grip 14 for the inner piston rod separately and FIG. 21 shows the locking element 15 for the inner piston rod separately.

The disclosed embodiments of the syringe show two chamber syringes, but the invention may obviously be applied to one or multiple chamber syringes and the number of yielding elements 16a-b then have to be adapted to the number of chambers. The yielding elements may, for multiple chamber syringes, be in fluid communication with the surroundings of the syringe directly or indirectly. Indirect fluid communication with the surroundings of the syringe means that expansion in a first yielding element a second yielding element, which is in fluid communication with the first, to expand, which in turn is in fluid communication with the surroundings of the syringe. The number of such yielding, elements that are connected via a may obviously be increased indefinitely in a corresponding way. In the claims, a yielding element that is in unspecified communication with the surroundings of the syringe means that this communication is indirect or direct.

1. A syringe comprising a cylindrical container in which is arranged at least a first which delimits at least one storage volume, wherein the first piston is provided with at least one yielding element having a first side that delimits the storage volume in a liquid tight fashion, and having a second side that is in communication with the surroundings of the syringe.

2. A syringe according to claim 1, wherein the yielding element resiliently strives to return to an unloaded position.

3. A syringe according to claim 2, wherein the yielding element is constituted by a bellows.

4. A syringe according to claim 1, wherein the yielding element is arranged on a first piston.

5. A syringe according to claim 1, wherein the yielding element with its second side is in communication with the surroundings of the syringe via a conduit in the first syringe.

6. A syringe according to claim 1, wherein the syringe comprises at least a first piston with a first piston. rod and a second piston with a second piston rod, where the first piston rod and the second piston rod may be locked together with a locking element.

7. A syringe according to claim 6, wherein the second piston is provided with a sealing element which in an unloaded state is sealing, while when loaded allows fluids to pass in a first direction, but when loaded disallows fluids to pass in a second direction.

8. A syringe according to claim 1, wherein the syringe is provided with piston rods that extend from the cylindrical container in a first direction and where the syringe at the end which faces in the opposite direction is arranged to receive a lid with a yielding element.
